(19) United States
(12) Patent Application Publication
(10) Pub. No.: US 2023/0364389 A1
(43) Pub. Date: Nov. 16, 2023

(54) Dressing with Integrated Securement System for Catheter

(71) Applicant: Becton, Dickinson and Company, Franklin Lakes, NJ (US)

(72) Inventors: Dorien Gemma Butter, Millcreek, UT (US); Jonathan Karl Burkholz, Salt Lake City, UT (US); John M. Lackey, West Valley City, UT (US); Todd L. Schwendiman, South Jordan, UT (US)

(21) Appl. No.: 18/196,206

(22) Filed: May 11, 2023

Related U.S. Application Data
(60) Provisional application No. 63/342,356, filed on May 16, 2022.

(51) Int. Cl. A61M 25/02

(52) U.S. Cl. CPC A61M 25/02; A61M 2025/024; A61M 2025/0266; A61M 2205/0205; A61M 2205/0238; A61M 2025/0246

(57) ABSTRACT

Provided herein is an integrated dressing for use with a catheter assembly, including a base defining a perimeter, the base comprising a bottom surface configured to contact skin of a patient, a cover portion attached to the base and configured to cover a site of a catheter insertion, and a stabilizing clip, attached to the base or the cover portion, and configured to releasably couple to a catheter adapter.

### CROSS-REFERENCE TO RELATED APPLICATION

The present application claims priority to U.S. Provisional Application Ser. No. 63/342,356 entitled “Dressing with Integrated Securement System for Catheter” filed May 16, 2022, the entire disclosure of which is hereby incorporated by reference in its entirety.

### BACKGROUND OF THE INVENTION

### Field of the Invention

Provided herein are devices and systems for use in vascular access, and, in particular, stabilization devices and vascular access systems including such devices.

### Description of Related Art

During dwell, a peripheral intravenous catheter (PIVC) may become occluded with thrombus or fibrin, preventing the ability to draw blood through the PIVC. Line draw devices are meant to pass through the catheter and open up a fluid path for blood draw. One challenge with these devices is successfully navigating through the s-shaped profile the catheter takes as it enters the vein. The catheter tubing may kink slightly at the bends of the s-curve (FIG. 2), preventing the line draw device from successfully passing though.

During insertion of a PIVC into a patient, the angle at which the catheter is inserted is typically around 30°, depending on a number of factors including the patient's vasculature, vein depth, and clinician technique. Once the catheter has been successfully inserted into the vein, the catheter adapter will be secured to the patient using adhesive tape or dressings. When securing the catheter adapter, the dressing is placed over the catheter adapter and the surrounding skin such that the catheter adapter lies roughly parallel to the skin. Because the catheter was inserted at a steeper angle relative to the skin, the tubing exiting the catheter adapter must transition from the plane of the adapter down into the vein, thus potentially kinking or deforming the tubing enough that a line draw device would be unable to pass through.

Proper securement of a PIVC is a critical step to ensure the success of line draw devices. Accordingly, there is a need in the art for a stabilization and securement device and dressing system to provide improved overall blood draw success, line draw device advancement reliability, and reduced risk of catheter complications resulting from additional access and use of the PIVC for blood collection.

### SUMMARY OF THE INVENTION

Provided herein is an integrated dressing for use with a catheter assembly, including a base defining a perimeter, the base comprising a bottom surface configured to contact skin of a patient, a cover portion attached to the base and configured to cover a site of a catheter insertion, and a stabilizing clip, attached to the base or the cover portion, and configured to releasably couple to a catheter adapter.

Also provided herein is a stabilized catheter set, including a catheter assembly including a catheter adapter, having a distal end, a proximal end, a lumen arranged between and in fluid communication with the distal end and the proximal end, and a catheter arranged at the distal end of the catheter adapter and in fluid communication with the lumen, and an integrated dressing, including a base defining a perimeter, the base comprising a bottom surface configured to contact skin of a patient, a cover portion attached to the base and configured to cover a site of a catheter insertion, and a stabilizing clip, attached to the base or the cover portion, and configured to releasably couple to a catheter adapter.

Also provided herein is a method of securing a catheter at a site of insertion, including the steps of providing a catheter assembly including a catheter adapter, having a distal end, a proximal end, a lumen arranged between and in fluid communication with the distal end and the proximal end, and a catheter arranged at the distal end of the catheter adapter and in fluid communication with the lumen, inserting the catheter into a patient, providing an integrated dressing including a base defining a perimeter, the base comprising a bottom surface configured to contact skin of a patient, a cover portion attached to the base and configured to cover a site of a catheter insertion, and a stabilizing clip, attached to the base or the cover portion, and configured to releasably couple to the catheter adapter, coupling the stabilizing clip to the catheter adapter, and securing the base to the skin of the patient.

### BRIEF DESCRIPTION OF THE DRAWINGS

FIG. 1 is a perspective view of a non-limiting embodiment of a catheter adapter useful with an integrated dressing as described herein;

FIG. 2 is a partial cross-sectional view of a conventional catheter adapter and catheter, showing the catheter inserted into a vein of a patient;

FIG. 3 is a bottom view of an integrated dressing according to a non-limiting embodiment described herein;

FIG. 4 is a perspective view of an integrated dressing and catheter adapter according to a non-limiting embodiment described herein; and

FIGS. 5A-5B are side views of an integrated dressing and catheter adapter according to a non-limiting embodiment described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein is a stabilization device for a catheter assembly. Stabilization devices as described herein may be used to provide a secure site of attachment of a catheter adapter to a site of a venipuncture, and may provide an optimal angle of entry of a catheter into the vasculature of a patient, reducing or eliminating kinking of medical devices that are passed through the catheter into the patient.

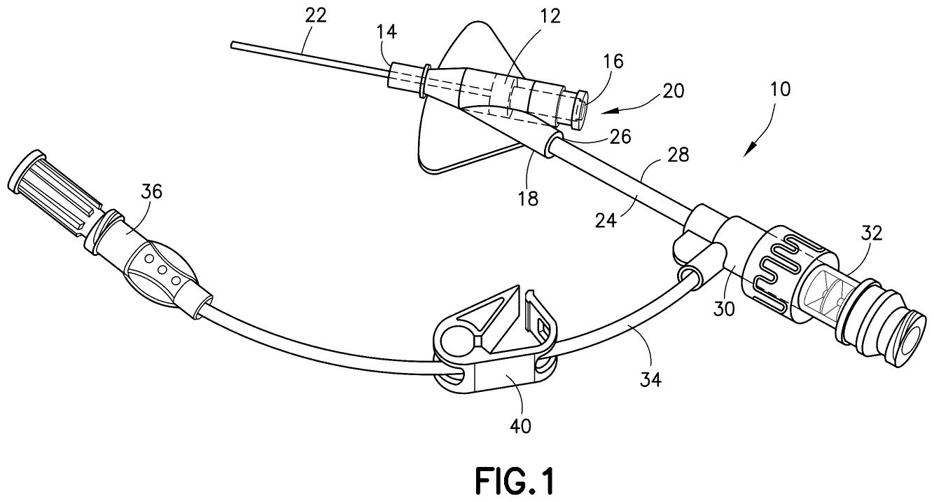

Referring now to FIG. 1, shown is a non-limiting embodiment of a catheter assembly 10 useful with stabilization devices as described herein below. Cather assembly 10 may include a catheter adapter 12, which may include a distal end 14 and a proximal end 16. In some embodiments, the catheter adapter 12 may include an additional port 18. In some embodiments, port 18 may disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, a 60, or a 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removably coupleable to catheter adapter 12, connector 30, and/or needless access connector 32) including a second fluid conduit, such as second fluid conduit 34. Extension sets 34 are known to those of skill in the art and are commercially available from, for example, Becton, Dickinson and Company under the tradename NEUTRACLEAR. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

Catheter assembly 10 may include a needleless access connector 32 and/or a second fluid conduit 34. Needleless access connectors 32 are known to those of skill in the art and are commercially available from, for example, Becton, Dickinson and Company under the tradenames MAXPLUS, MAXZERO, Q-SYTE, and SMARTSITE.

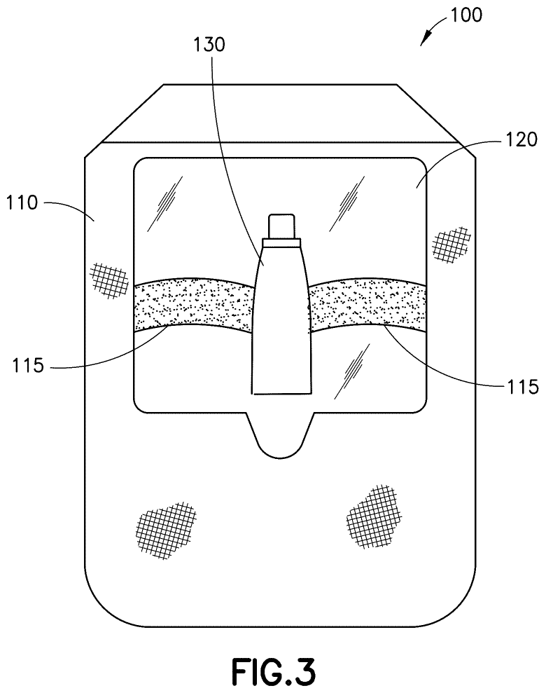

Turning to FIG. 3, shown is a non-limiting embodiment of an integrated dressing 100, including a base 110 and a cover portion 120. Base 110 may be formed of any suitable material known to those of skill in the art, including fabrics. Base 110 may define a perimeter of any suitable shape. In non-limiting embodiments, base 110 includes a top surface and a bottom surface, the bottom surface configured to contact skin of a patient onto which integrated dressing 100 is applied. In non-limiting embodiments, base 110 includes one or more suitable adhesives known to those of skill in the art on at least a portion of the bottom surface. In some non-limiting embodiments, portions of base 110 that include an adhesive are covered by removable tabs 112. In non-limiting embodiments, the one or more adhesives may include one or more antimicrobial compositions. Suitable antimicrobial compositions are known to those of skill in the art and may include, for example, chlorhexidine gluconate (CHG).

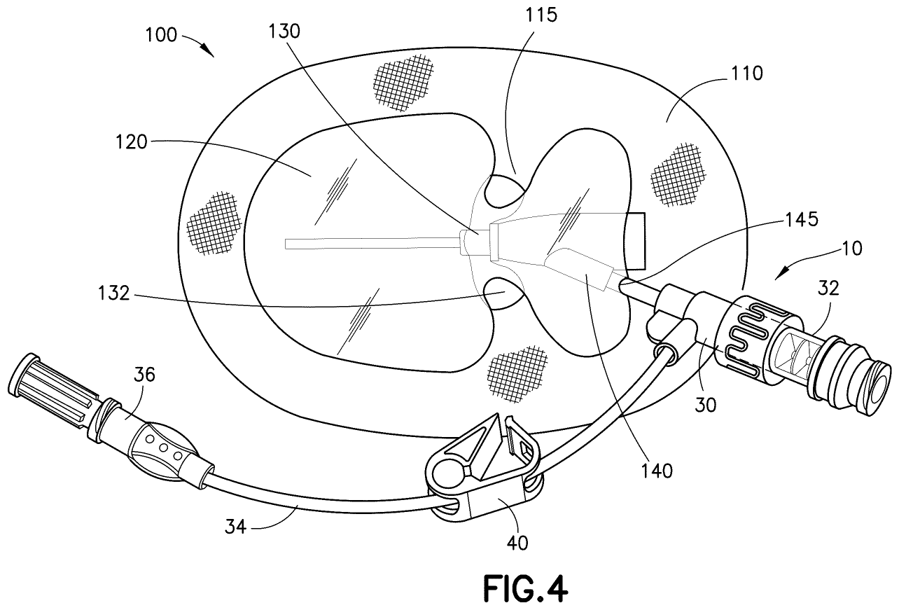

With continuing reference to FIG. 3, and with reference to FIG. 4, integrated dressing 100 also includes a cover portion 120 coupled to base 110. Cover portion may be coupled to base 110 at one or more locations along the perimeter defined by base 110, or substantially continuously along the perimeter. In some non-limiting embodiments, as shown in FIG. 4, cover portion 120 may include an opening 145 to allow at least a portion of a catheter assembly, such as catheter assembly 10 shown in FIG. 1, to pass therethrough. In some non-limiting embodiments, opening 145 is configured to allow first fluid conduit to pass therethrough. Cover portion 120 may be a single portion, as shown in FIG. 3, or may be divided into a plurality of portions 120, 140, for example as shown in FIG. 4. In some non-limiting embodiments, cover portion 120, 140 is substantially or completely transparent, to allow for visualization of catheter assembly 10 arranged thereunder.

Integrated dressing further may further include stabilizing clip 130, which is configured to couple, optionally reversibly, to one or more portions of a catheter adapter, such as catheter adapted 12. In some non-limiting embodiments, stabilizing clip 130 couples to distal end 14 of catheter adapter 12. In some non-limiting embodiments, stabilizing clip 130 couples to catheter adapter 12 by snap fit, press fit, friction fit, or other connection known to those of skill in the art. In some non-limiting embodiments or aspects, stabilizing clip is formed of a plastic, elastomeric material, or other resilient material. In some non-limiting embodiments, stabilizing clip 130 is biased towards a position that secures one or more parts of catheter adapter 12 therein when coupled thereto, e.g., one or more portions of stabilizer clip 130 must be moved away from one another to allow for a part of catheter adapter 12 to couple to stabilizer clip 130. In some non-limiting embodiments, stabilizer clip 130 is coupled to base 110, for example by one or more arms 115. One or more arms 115 may be formed as part of base 110, and/or may be formed of the same material as base 110.

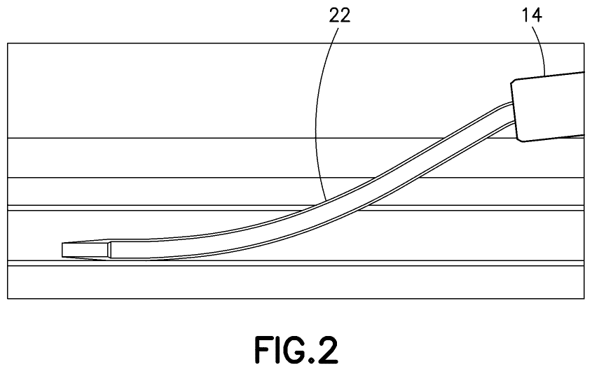

Stabilizing clip 130 may be configured to hold catheter adapter 12 at a certain angle, or within a certain range of angles, relative to the skin of the patient. Stabilizing clip thus may be angled relative to base 110, such that catheter adapter 12 is arranged at an angle of between about 3 degrees and about 10 degrees (all values and subranges therebetween inclusive), optionally between about 6 degrees and about 10 degrees, optionally about 6 degrees, relative to skin of a patient onto which integrated dressing 100 is applied. Stabilizer clip 130 may include one or more features to maintain an optimal “s” curve of catheter 12 (e.g., FIG. 2) while it is inserted into patient, and, in addition, may optionally include one or more features to provide a downward flex to the catheter and/or provide strain relief therefor

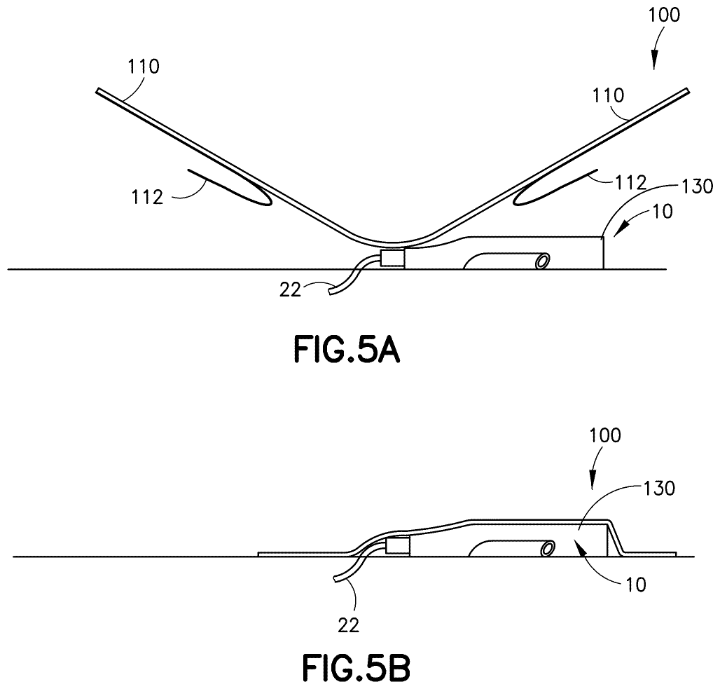

Turning to FIGS. 5A-5B, shown is a non-limiting embodiment of a method of securing a catheter assembly to a patient with an integrated dressing 100 as described herein. In some non-limiting embodiments, a catheter, such as catheter 22 as part of catheter assembly 10 illustrated in FIG. 1, is inserted into skin of a patient. Stabilizing clip (not shown in FIGS. 5A-5B) is then coupled to catheter adapter 12 (FIG. 5A), for example by pressing stabilizing clip onto catheter adapter 12. Integrated dressing may then be applied, for example by removing tabs 112 that cover portions of base 110 that include adhesive, allowing for securement of integrated dressing 100 to the patient's skin (FIG. 5B). Use of the integrated dressing 100 as described herein can maintain catheter at a proper angle to allow for ease of insertion of various devices through catheter into vasculature, and can maintain sterility of the insertion site.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An integrated dressing for use with a catheter assembly, comprising:
a base defining a perimeter, the base comprising a bottom surface configured to contact skin of a patient;
a cover portion attached to the base and configured to cover a site of a catheter insertion; and
a stabilizing clip, attached to the base or the cover portion, and configured to releasably couple to a catheter adapter.

2. The integrated dressing of claim 1, wherein the base comprises a fabric.

3. The integrated dressing of claim 2, wherein the fabric comprises an adhesive on the bottom surface thereof.

4. The integrated dressing of claim 1, further comprising a peelable tab arranged on the bottom surface of the base, covering the adhesive.

5. The integrated dressing of claim 1, wherein the base further comprises one or more arms extending inward from the perimeter, and wherein the stabilizing clip is coupled to the one or more arms.

6. The integrated dressing of claim 1, wherein the stabilizing clip is configured such that the catheter adapter is maintained at an angle relative to the skin of the patient.

7. The integrated dressing of claim 6, wherein the stabilizing clip is configured such that the catheter adapter is maintained at an angle of about 3 degrees to about 10 degrees relative to the skin of the patient.

8. The integrated dressing of claim 1, wherein the cover portion comprises an opening configured to allow at least a portion of the catheter assembly to pass therethrough.

9. A stabilized catheter set, comprising:
a catheter assembly comprising:
a catheter adapter, comprising:
a distal end;
a proximal end;
a lumen arranged between and in fluid communication with the distal end and the proximal end; and
a catheter arranged at the distal end of the catheter adapter and in fluid communication with the lumen; and
an integrated dressing, comprising.
a base defining a perimeter, the base comprising a bottom surface configured to contact skin of a patient;
a cover portion attached to the base and configured to cover a site of a catheter insertion; and
a stabilizing clip, attached to the base or the cover portion, and configured to releasably couple to the catheter adapter.

10. The stabilized catheter set of claim 9, wherein the base comprises a fabric.

11. The stabilized catheter set of claim 10, wherein the fabric comprises an adhesive on the bottom surface thereof.

12. The stabilized catheter set of claim 9, further comprising a peelable tab arranged on the bottom surface of the base, covering the adhesive.

13. The stabilized catheter set of claim 9, wherein the base further comprises one or more arms extending inward from the perimeter, and wherein the stabilizing clip is coupled to the one or more arms.

14. The stabilized catheter set of claim 9, wherein the stabilizing clip is configured such that the catheter adapter is maintained at an angle relative to the skin of the patient.

15. The stabilized catheter set of claim 14, wherein the stabilizing clip is configured such that the catheter adapter is maintained at an angle of about 3 degrees to about 10 degrees relative to the skin of the patient.

16. The stabilized catheter set of claim 9, wherein the cover portion comprises an opening configured to allow at least a portion of the catheter assembly to pass therethrough.

17. A method of securing a catheter at a site of insertion, comprising:
providing a catheter assembly comprising:
a catheter adapter, comprising:
a distal end;
proximal end;
a lumen arranged between and in fluid communication with the distal end and the proximal end; and
a catheter arranged at the distal end of the catheter adapter and in fluid communication with the lumen;
inserting the catheter into a patient;
providing an integrated dressing comprising:
a base defining a perimeter, the base comprising a bottom surface configured to contact skin of a patient;
a cover portion attached to the base and configured to cover a site of a catheter insertion; and
a stabilizing clip, attached to the base or the cover portion, and configured to releasably couple to the catheter adapter;
coupling the stabilizing clip to the catheter adapter; and
securing the base to the skin of the patient.

18. The method of claim 17, wherein the integrated dressing further comprises an adhesive on the bottom surface thereof and a peelable tab arranged on the bottom surface of the base, covering the adhesive, and wherein the method further comprises, prior to securing the base to the skin of the patient, removing the peelable tab from the bottom surface of the base.

19. The method of claim 17, wherein the bottom surface of the base further comprises an antimicrobial coating.

20. The method of claim 17, wherein, when secured, the catheter adapter is maintained at an angle of about 3 degrees to about 10 degrees relative to the skin of the patient.
